(19) United States
(12) Patent Application Publication
(10) Pub. No.: US 2006/0222722 A1
(43) Pub. Date: Oct. 5, 2006

(54) Sublingual methods of treatment to alleviate or prevent arthritis

(76) Inventors: Stephen C. Roberts, Minnetonka, MN (US); Douglas P. Root, St. Paul, MN (US)

Correspondence Address: GelStat Corporation;Southpoint Office Center, 1650 West 82nd Street, Suite 1040, Bloomington, MN, 55431, US

(21) Appl. No.: 11/388,693

(22) Filed: Mar. 24, 2006

Related U.S. Application Data
(60) Provisional application No. 60/666,881, filed on Mar. 30, 2005.

(51) Int. Cl. A61K 36/906; A61K 36/28

(52) U.S. Cl. 424/756; 424/764

(57) ABSTRACT

Methods of treating arthritis and associated symptoms are provided by administering a composition comprising feverfew extract and ginger extract sublingually to a patient in need thereof. Treatments are surprisingly effective with low total administered amounts of feverfew extract and ginger extract.

This application claims the benefit of U.S. Provisional Application Ser. No. 60/666,881, filed Mar. 30, 2005, entitled “SUBLINGUAL METHODS OF TREATMENT TO ALLEVIATE OR PREVENT ARTHRITIS” which application is incorporated herein by reference in its entirety.

### FIELD OF THE INVENTION

The present invention relates to treatment of arthritis and associated symptoms. The present invention also relates to sublingual treatment regimens and compositions comprising feverfew extract and ginger extract.

### BACKGROUND OF THE INVENTION

Arthritis is a collective term for number of different conditions that cause pain, swelling and limited movement in joints and connective tissue throughout the body. Specific causes for arthritis are not yet known for most forms of the disease. The condition is usually chronic. The main symptoms of arthritis are joint pain, joint stiffness or inability to move a joint normally, and sometimes swelling that lasts more than two weeks. Most treatment programs include a combination of medication, exercise, rest, use of heat and cold, joint protection techniques, and sometimes surgery. Additional treatments include administration of various medicaments, such as nonsteroidal anti-inflammatory drugs (herein after NSAIDs) including aspirin, ibuprofen, and naproxen for pain relief and inflammation reduction, and corticosteroid injection directly into affected joints in acute cases and administration of steroids, such as disclosed in U.S. Pat. No. 6,794,374.

Evaluation of various non-traditional medical approaches has taken place to address this disease. For example, a double-blind, placebo-controlled study was carried out in Nottingham, U.K. in 1989 to test the efficacy of feverfew in treating the symptoms of rheumatoid arthritis. Patrick M, Heptinstall S, et al. Feverfew in rheumatoid arthritis: a double-blind, placebo-controlled study. Annal Rheu Dis. 1989:48:547-549. Forty-one female patients were given either dried leaf capsules (700 mg) or a placebo once a day for six weeks. No relief from pain or joint stiffness was seen in the feverfew treatment group.

U.S. Pat. No. 4,758,433 Johnson, et al., issued Jul. 19, 1988 discloses a preparation for pharmaceutical use, especially in the treatment of migraine, arthritis and bronchial complaints that contains a sesquiterpene lactone and is recovered from the plant Tanacetum parthenium by extraction using a pharmaceutically acceptable oil and is also delivered via the pharmaceutically acceptable oil, the oil being selected from the group consisting of saturated and un-saturated long chain hydrocarbons and fatty acids, vegetable and animal oils and polyoxyethylated derivatives thereof and reconstituted glycerides and esters thereof, wherein the hydrocarbon chain comprises between 10 and 25 carbon atoms, especially coconut oil, soybean oil or fish oil.

Both feverfew and ginger have a long history of use as homeopathic and herbal remedies. Ginger has been used with some success for relief of nausea. The administration of 1,000 to 2,000 mg of ginger orally by tablet has been found to effectively reduce nausea in the case of motion sickness (Lien, HC Am J Physiol Gastrointest Liver Physiol March 2003; 284(3):G481-9). Ginger was investigated recently for its effect on nausea in morning sickness (Keating, A., Altern Ther Health Med September-October 2002; 8(5):89-91). In this study women took 1,000 mg of ginger in divided doses, the researchers concluding that ginger “may be helpful” in treatment of morning sickness. According to the Physician's Desk Reference for Herbal Medicines, the daily recommended dose of ginger for a variety of gastrointestinal symptoms (primarily various forms of nausea) ranges from 500 mg to 4000 mg. (PDR for Herbal Medicines, Thompson Medical Economics, Second Edition, Ginger, 339-342, 2000.)

Feverfew is an herb that is widely available and has been investigated in modem times. Historically, feverfew is known to have been used in the treatment of fevers, from whence it derives its name, and also in rheumatic conditions. Feverfew is used in homeopathic remedies, but homeopathy recognizes no role for feverfew in the treatment of headaches. An authoritative homeopathic text is “A Dictionary Of Practical Materia Medica” by John Henry Clarke, M. D., recognized as such by the United States Food and Drug Administration, (see the “Compliance Policy Guide: Conditions Under Which Homeopathic Drugs may be Marketed”, located on the internet under the FDA website. This text defines the appropriate preparation of feverfew as “a tincture of fresh leaves.” A tincture is a concentrated herbal extract prepared by soaking an herb in alcohol for an extended period of time. The result is an alcoholic extract referred to by homeopathic practitioners as the “mother tincture.” This “mother tincture” is then subject to numerous serial dilutions with the resulting homeopathic drug being extremely dilute. Classic homeopathic remedies do not rely on any effect from the substance first contained in the starting material (“mother tincture” in this case) and often statistically contain virtually no actual molecules of the original substance (feverfew). Instead, these remedies rely on the “imprint” or “energy” of the original substance to exert an effect. Consistent with the precepts of homeopathy, the remedy thus prepared is felt to become increasingly potent, indeed stronger and more effective, as it becomes more and more dilute. Some of these homeopathic remedies may have been administered sublingually, or may yet be administered sublingually by those presently adherent to the practice.

Herbal medicine, as a field distinct from classical homeopathy, has recognized the potential value of feverfew in the prophylactic (preventative) treatment of migraine. Fresh feverfew leaves have sometimes been chewed by subjects wishing to rid themselves of migraine. However, a common adverse effect reported by those who have used this technique is the generation sores in the mouth and sensitization of oral tissues. Additionally, many patients find this mode of administration to be crude and unpleasant.

In addition to raw leaves, feverfew tablets or capsules have been and are employed by practitioners of herbal medicine. These are widely available in any “health food store” for purchase by the general public. The PDR for Herbal Medicines lists migraine, arthritis, rheumatic diseases and allergies as the indications for feverfew usage (PDR for Herbal Medicines, Thompson Medical Economics, Second Edition, Feverfew, 306-309, 2000.)

U.S. Pat. No. 6,841,544 discloses preparations for treating the symptoms of colds, flu, allergies, or sinus discomfort as well as treating pain and discomfort associated with heartburn, general body aches, headaches, migraines, menstruation, joint discomfort and arthritis. These preparations are a combination of synthetic pharmaceutical ingredients together with neutraceuticals. The pharmaceutical ingredients, are “preferably selected from a group which includes, for example, acetaminophen, acetylsalicylic acid or an effective salt thereof, ibuprofen, ketoprofen, naproxen, naprosyn phenylpropanolamine bitartarate or an effective salt thereof, pseudoephedrine hydrochloride or an effective salt thereof, diphenhydramine hydrochloride or an effective salt thereof, clemastine fumarate or an effective salt thereof, chlorpheniramine maleate or an effective salt thereof, bromopheniramine maleate or an effective salt thereof, guaifenesin, dextromethorphan hydrochloride or an effective salt thereof, dextromethorphan hydrobromide or an effective salt thereof, famostidine, ranitidine, cimetidine, phenindamine tartarate or an effective salt thereof, calcium carbonate or an effective salt thereof, and combinations thereof.” The nutraceutical ingredients are disclosed to be “preferably selected from the group which includes, for example, Echinacea purpurea, Echinacea angustifolia, Echinacea pillida, Gingko biloba, saw palmetto, ginseng, cat's claw (una de gato), cayenne, bilberry, cranberry, grapeseed extract, St. John's wort, cascara sagrada, valerian, elderberry, elder flower, sweet elder, Sambucous nigra, Sambucous canadensis, garlic, Camellia sinensis, Camellia thea, Camellia theifera, Thea sinensis, Thea bohea, Thea viridis, goldenseal, wild cherry (Rosacea), quercetin, stinging nettles (Urtica), curcumin, bromelain, multiple pancreatic enzymes (protease, protease II, protease m, peptidase, amylase, lipase, cellulase, maltase, lactase, invertase), Emblica officinalis, eicosapentaenoic acid, docosahexaeonic acid, primrose oil, feverfew, ginger root, vitamin E (D-alpha-tocopherol), licorice root (Glycyrrhiza uralensis), aloe vera, horseradish root, L-glutamine, ascorbic acid, antiscorbutic vitamin, rose hips, calcium ascorbate, cevitamic acid, citrus bioflavonoids complex, acerola, zinc or an effective salt thereof, Astragalus membranaceous, Astragalus mongolicus, membranous milk vetch, milk vetch, mongolian milk, dong quai, huangqi, hunag qi, moringa and combinations thereof. Although these ingredients are preferred, other pharmaceuticals and nutraceuticals may be substituted in their place.” These preparations are taught to be provided by “compounding them into a pharmaceutically acceptable dosage form.” The pharmaceutical dosage forms contemplated by this disclosure include tablets; capsules; orally-administered or injectable liquids, suspensions, or dispersions; powders; suppositories; and a nasal decongestant spray. This disclosure does not contemplate sublingual administration.

Additionally, it is noteworthy that the content of various measurable ingredients, such as parthenolide, in feverfew may vary to a great extent depending on the particular variety of Tanacetum parthenium plant grown, and also the manner of processing the feverfew herb. The collection and processing steps carried out incorporating feverfew extract into a product may reduce or destroy the active ingredient content of the feverfew. Without a standardization of the marker chemical, such as parthenolide, during the initial manufacturing, great inconsistency of ingredient content may be observed from batch to batch of product.

### SUMMARY OF THE INVENTION

The present invention provides a method of treating arthritis comprising sublingually administering an effective amount of a composition comprising feverfew extract and ginger extract to a subject in need of such treatment.

In a preferred aspect of the present invention, the composition comprising feverfew extract and ginger extract is provided in a sublingual tablet formulation. In this embodiment, the tablet comprises feverfew extract and ginger extract, as well as other ingredients in the formulation to provide a tablet suitable for administration of the feverfew extract and ginger extract sublingually. Preferably, the tablet comprises components that facilitate rapid disintegration of the tablet under sublingual conditions of use.

In an alternative aspect of the present invention, the composition comprising feverfew extract and ginger extract is provided in a sublingual liquid or gel format. In other alternative aspects of the present invention, the composition comprising feverfew extract and ginger extract is provided in alternative formats, such as dissolvable films.

The method of the present invention provides surprisingly effective treatment of arthritis with a relatively small amount of active ingredient. Most beneficially, the method of the present invention provides arthritis treatment using over the counter medications that are well tolerated by patients. Surprisingly, effective treatment can be realized in a short time period, and additionally or alternatively prophylactic treatment of arthritis can be obtained by highly effective administration of small amounts of well tolerated ingredients on a regular basis with little or no concern regarding adverse narcotic or other effects common to prescription-type medications.

In particular, the combination of feverfew extract together with ginger extract is particularly effective in providing relief from arthritis when provided and administered as taught herein. It has been observed that the combination of these extracts in the present compositions in particular provide synergistic effect in relieving both the pain and the general discomfort associated with arthritis that is beyond a mere additive effect of these extracts. Incorporation of additional herbal extracts in amounts as indicated herein, when delivering the composition as a sublingual aqueous administration, also provide excellent total beneficial effects of the composition exceeding what would be expected from the herbal and homeopathic literature.

### DETAILED DESCRIPTION

It has surprisingly been found that administration of a very small amount of feverfew extract and ginger extract sublingually provides excellent treatment for arthritis, including relief of arthritis inflammation and other symptoms (i.e. pain, swelling, restricted movement, etc.). Arthritis treatment methods in accordance with the present invention preferably are administered in a manner in order to improve movement and comfort of a patient in need thereof.

For purposes of the present invention, “Treating” or “treatment” of a disease includes:
(1) preventing the disease, i.e., causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, ie., causing regression of the disease or its clinical symptoms.

The term “therapeutically effective amount” means the amount of the subject compound that will elicit the biological or medical response of a tissue, system, animal or human that is being sought by the researcher, veterinarian, medical doctor or other clinician.“A therapeutically effective amount” includes the amount of a compound that, when administered to a mammal for treating a disease, is sufficient to effect such treatment for the disease. The “therapeutically effective amount” will vary depending on the compound, the severity of the disease, and the age, weight, etc., of the mammal to be treated.

The ability to effectively treat arthritis and the associated symptoms using such low yet rapidly effective doses of active ingredient provides substantial benefits, including not only effective relief from arthritis and their associated symptoms, but also the drastic reduction in the side effects which might otherwise be associated with the active ingredient. By far the most commonly reported side effect of feverfew use is the occurrence of mouth sores, primarily associated with chewing feverfew leaves, wherein it may be observed in up to 11% of such users. Mouth sores are occasionally accompanied by general inflammation of tissues in the mouth. Infrequently reported side effects of feverfew use include gastrointestinal side effects such as abdominal pain, indigestion, flatulence, diarrhea, nausea, and vomiting. The methods and compositions of the present invention thus may provide an improved safety profile that may make the present invention most suitable to the average user, and particularly suitable for those with whom additional caution need be exercised, such as those who are sensitive to various medications or in the case of pediatric use, where additional cautions are generally warranted. The invention is particularly beneficial to those patients concerned about using large amounts of medication for treatment of ailments, and also to patients who wish to avoid the use of prescription medications, or who cannot afford the use of prescription medications. The low total administered amount of active ingredient and relatively small amount of total composition that is applied sublingually may additionally be of particular benefit to those treating arthritis. The preparation is further beneficial to those wishing to employ arthritis prophylaxis from time to time without the need to maintain daily intake of medication indefinitely.

As a new over-the-counter (“OTC”) medication, the present invention provides substantial benefits to persons suffering from arthritis, not least of which may be the cost savings associated with a decreased reliance on expensive prescription medications and the reduction in the economic burden of arthritis in the United States. There are also substantial advantages for those individuals who choose to rely on OTC medications for relief of arthritis, namely the availability of another treatment option that may be more effective than currently available OTC products, or which may be associated with fewer side effects than currently available OTC products.

Presently known products are generally slow in providing relief, and may often exhibit undesired side effects. Surprisingly, the present invention can provide rapid relief of symptoms—in some cases beneficial effects of the treatment are felt within hours of administration. In other cases, relief can be observed in a matter of only a few days of administration of small doses of feverfew extract and ginger extract. This administration over time of small doses surprisingly can be more effective than single large doses of the same components.

As in many diseases, the treatments for arthritis can be very patient specific in effectiveness. The search for effective treatment can be a lengthy and sometimes costly search by each person for the treatment that will be effective for that particular individual. Surprisingly, the present invention is effective for a large percentage of people who try this treatment. Thus, the present invention provides additional substantial benefit to the practitioner or individual who may discover conveniently and without delay those for whom this treatment is effective, searching amongst other alternatives in those cases where this medication proves insufficiently effective.

Additionally, there is a growing desire to use naturally generated ingredients to provide treatments for disorders. Therefore, a particularly preferred embodiment of the present invention comprises administration of small quantities of feverfew extract and ginger extract. More specifically, a preferred method of treating symptoms of arthritis comprises sublingually administering a composition provided in unit dose form, wherein the unit dose form comprises from about 0.1 mg to about 5 mg of feverfew extract and from about 1 mg to about 20 mg of ginger extract per unit dose. In a more preferred embodiment, the unit dose form comprises from about 0.1 mg to about 3 mg of feverfew extract and from about 3 mg to about 10 mg of ginger extract per unit dose. In a preferred embodiment of the present method, from about 1 to about five unit doses as described above are administered in a twenty-four hour period. Thus, a very low total overall dose of feverfew extract and ginger extract is preferably administered to the patient.

Feverfew extract is derived from the feverfew plant (Tanaecetum parthenium), which is also known, for example, as Chrysanthemum parthenium, Chrisanthemum parthenium, Pyrethrum parthenium, Tanacete parthenii herba or folium, Matricaria parthenoides, Matricaria parthenium, Leucanthemum parthenium, Matricaria parthenium, Spanish pellitory, Featherfew, Featherfoil, feather-fully, and by a number of common names, various of which are used throughout the world (Midsummer daisy, Bachelor's buttons, Altamisa, nosebleed, flirtwort, ague plant, devil daisy, feddygen fenyw (Welsh), maid's weed, Missouri snakeroot, mutterkaut (German), prairie-dock, vetter-voo, wild chamomile, grande camomille (French), Santa Maria (Spain), febrifuge plant.) The extract may be obtained by techniques known in the art using solvents such as petroleum spirits or polar organic solvents. See U.S. Pat. No. 5,384,121 to Rhodes, and also WO 94 06800; EP 0 553 658; WO 92 11857; GB 2,166,952; EP 98 041; WO 98 39018.

The extract of the feverfew plant at least initially contains parthenolide, and may additionally contain other components such as Polyynes, Flavonoids and Volatile oils including camphor, bomeol and others, each of which may contribute to the therapeutic effect of the preparation disclosed herein. Feverfew also contains relatively large quantities of sesquiterpene lactones, primarily parthenolide.

In addition to parthenolide, feverfew is known to contain the following non-ubiquitous chemicals: 1-Beta-hydroxyarbusculin, 10-Epicanin, 8-Beta-reynosin, Apigenin-7-glucoside, Chrysanthemolide, Chrysanthemonin, Chrysartemin-A, Chrysartemin-B, Cosmosiin, L-Bomeol, L-camphor, Mangoliolide, Reynosin, Santamarin, Tanaparthin, Tanaparthin-1-alpha, 4-alpha-epoxide, Tanaparthin-1-beta, 4-beta-epoxide, tenetin 3-b-hydroxyparthenolide, seco-tanaparthenolide A, canin, artecanin, and balchanin.

Because feverfew extract may contain additional beneficial components, compositions comprising the extract of feverfew are generally preferred for use in the present invention as compared to compositions comprising a highly purified parthenolide that has been isolated from the additional components naturally occurring in feverfew extract. Preferred embodiments of the present invention use feverfew extract that has been standardized at initial manufacture to contain a predetermined standardized parthenolide concentration of preferably not less than about 0.5%, and more preferably from about 0.8% to about 2.0%. Most preferably, the feverfew extract has been standardized at initial manufacture to contain a predetermined standardized parthenolide concentration of about 1%.

Likewise, ginger extract is derived from the ginger root, and may contain beneficial components in addition to gingerols, the generally recognized components of ginger extract. Thus, preferred embodiments of the present invention use ginger extract that has been standardized at initial manufacture to contain a predetermined standardized gingerol concentration of preferably not less than about 1%, more preferably from about 2 to about 20%, and most preferably from about 2% to about 6%. In a particularly preferred embodiment, the ginger extract has been standardized at initial manufacture to contain a predetermined standardized gingerol concentration of about 4%.

The composition to be used in the present invention may optionally comprise additional active ingredients. These active ingredients may also be provided as a treatment of arthritis or may provide other physical benefits, provided that the treatment benefit of the feverfew extract is not adversely affected. In one aspect, preferably additional amounts of already present sesquiterpene lactones or additional sesquiterpene lactones are incorporated in the compositions of the present invention. Preferred such sesquiterpene lactones include especially those which are known to be contained in (naturally occur in) feverfew, such as 3-beta-hydroxyparthenolide, seco-tanaparthenolide A, canin, artecanin, chrysanthemonin, chrysartemin A and B, santamarin and balchanin, as well as those occurring in other plant species such as encelin, leucanthin B, enhydrin, melampodin A, tenulin, confertiflorin, burrodin, psilostachyin A, costunolide, guaianolide, cinerenin, artemisinin, aristolactone, lactarorufin A, bilobalide, helenalin, furandiol. Sesquiterpene lactones in addition to parthenolide may be isolated from plants such as dandelion, burdock, butterburr, mugwort and sunflower plants, among others.

Compositions to be used in the present invention may optionally additionally comprise other naturally occurring components and extracts. Preferred additional components are extracts indicated for use in treatment of inflammation, nausea or anxiety.

Particularly preferred compositions of the present invention contain substantially no active ingredients other than those that are extractable from herbal sources. In a particularly preferred embodiment of the present invention, the compositions contain substantially no active ingredients other than those that are extractable from feverfew, ginger and green tea sources. In another particularly preferred embodiment, the compositions contain substantially no active ingredients other than those that are extractable from feverfew and ginger. Such compositions additionally may comprise non-pharmacologically active ingredients, such as thickeners, carrier liquids and flavorants. It has surprisingly been discovered that the use of only active ingredients that are extracted from herbs provide particular benefit to the user in being both effective in the treatment of arthritis, and also providing natural healing conditions particularly suited to the well being of patients. Surprisingly, these natural ingredients have been found to be effective in the indicated dosage ranges when administered in a sublingual regimen as described herein. Such compositions contain the feverfew extract and ginger extract in the amounts as discussed earlier, and preferably contain less than about 400 mg of any given natural active ingredient per dose.

The compositions as described herein may further comprise suitable adjuvants, such as preservatives (for example, sodium benzoate, sorbic acid and esters of p-hydroxybenzoic acid), stabilizers, antibacterial agents (such as benzyl alcohol or methyl paraben), antioxidants (such as ascorbic acid or sodium bisulfite), chelating agents (such as ethylenediaminetetraacetic acid), buffers (such as acetates, citrates or phosphates), agents for the adjustment of tonicity (such as sodium chloride or dextrose), dyes, colorants, thickening agents, flavorants, sweetening agents, and suspending agents.

In a particularly preferred embodiment of the present invention, the compositions of the present invention are provided in combination with a mucosal permeation enhancer appropriate for enhancing the mucosal absorption of the composition employed. The mucosal permeation enhancer preferably comprises azone, sodium glycholate, sodium cholate, sodium taurocholate, sodium taurocholate plus EDTA, deoxycholate, sodium lauryl sulfate, lauric acid, ethanol, lysophosphatidyl choline, polysorbate 80, cyclodextrin, cetylpyridinium chloride, cetyltrimethylammonium bromide, benzalkonium chloride, sodium salicylate, sodium EDTA, aprotinin, dextran sulfate, linoleic acid, labrafil, transcutol, urea, methoxysalicylate, POE 23 lauryl ether, various surfactants and other mucosal permeation enhancers and combinations thereof. Most preferably, the mucosal permeation enhancer comprises sodium lauryl sulfate.

Preferably, the composition is buffered by a pharmaceutically acceptable buffer. Examples of buffering agents include borate buffers, citrate buffers, phosphate buffers, tartarate buffers, acetate buffers, carbonate buffers, and amino acid salts, etc. Most preferably, the buffer is sodium citrate.

As noted above, the composition for use in the present method can be provided in any format suitable for sublingual administration.

In a particularly preferred format, the composition is provided in tablet or lozenge form. In one aspect, the tablet is provided in a rapid-dissolving form to facilitate rapid administration of components contained therein to the sublingual environment. In a preferred aspect, the tablet is formulated using techniques well known to the skilled artisan to provide a tablet that disintegrates in water in from about 10 to about 200 seconds. It will be readily appreciated that during sublingual administration, the tablet would disintegrate under the tongue in a similar timeframe. Quick dissolving tablets may be formed, for example, from a particulate support matrix containing the therapeutic agent, where the particulate support matrix is a protein (U.S. Pat. Nos. 5,807,576, 5,635,210, 5,595,761). Alternatively, the tablet may be formed from a laminate with several layers and an outer coating (JP 100535518). Tablets have also been manufactured from shearform matrices, which are substantially amorphous sugar formed when crystalline sugar is subjected to heat and shear (WO 95/07194; WO 95/35293). Other methods of forming quick dissolving tablets include wet granulation methods (EP 0627 218) and dry granulation methods (EP 0124027A1) and by freeze-drying techniques (EP 0084705A2).

In an alternative format, compositions for use in the present invention may be formulated in the form of fast-dissolving films in a manner that will now be apparent to the skilled artisan. Films that incorporate a active ingredients are disclosed, for example, in U.S. Pat. No. 4,136,145 to Fuchs, et al., U.S. Pat. No. 4,849,246 to Schmidt, U.S. Pat. No. 5,629,003 to Horstmann et al. and U.S. Pat. No. 5,948,430 to Zerbe et al. and U.S. Patent Application No. 2005/0037055 filed by Yang, et al.

In yet another alternative format, compositions for use in the present method may be provided in liquid or gel form. In this embodiment, liquid compositions as described herein are formulated using a carrier liquid appropriate for administration to the sublingual region of the mouth. The carrier liquid preferably is selected from water, alcohol, polyethylene glycols, glycerin, propylene glycol, and mixtures thereof. Most preferably the carrier liquid comprises water.

Thickening agents are preferably incorporated in liquid/gel compositions for use in the present invention. The thickening agent preferably assists in retention of the liquid composition sublingually for a time sufficient to allow absorption of the active ingredients in by the patient. Thickening agents are particularly desirable in sublingual applications, as a more viscous agent is more easily retained in the proper area. A more viscous agent further reduces the user's involuntary impulse to swallow, in this case perhaps prematurely. Thus, the thickening agent may assist in providing sublingual liquid retention for a time appropriate for proper absorption of the active ingredient by the patient, and also thereby may improve the clinical efficacy of the composition. Any appropriate thickening agent may be used in the composition of the present invention. Preferred such thickening agents include agar, alginate, carageenan, carboxymethylcellulose, cellulose, chitosan, corn starch, Danish agar, dextrin, furcelleran, galactomannans, gelatin, gellan gum, guar gum, gum acacia, gum arabic, gum ghatti, gum tragacanth, hydroxypropyl methylcellulose, karaya gum, methylcellulose, polyvinyl alcohol, carboxyvinyl polymer, polyvinylpyrrolidone, hyaluronic acid and salts thereof, modified starches, mucilage, pectin, potato starch, rice starch, starch, tara gum, vegetable starch, wheat starch, and xanthan gum and combinations thereof.

Most preferably, the liquid/gel compositions for use in the present invention have a viscosity that is from about 100 cP (somewhat lower than the viscosity of Olive Oil) to about 50,000 (i.e. the viscosity of molasses), and more preferably from about 500 cP (the viscosity of SAE #10 motor oil) to about 5000 cP (approximately the viscosity of Corn Syrup), all measured at 25° C.

Compositions provided in liquid or gel form as described herein may be administered using any appropriate technique, such as by use of a medicine dropper, syringe, vial, or the like. Most preferably, the aqueous composition is administered in a controlled manner as a flow of liquid, rather than as a spray. A flowing liquid dispenser provides benefits of controlled delivery of the liquid to the desired position in the mouth, enhancing the likelihood that the composition to be dispensed is properly delivered. Preferably, the composition is administered using a unit dose applicator that is a dispenser having a reservoir and a delivery spout and having a liquid capacity of about 0.1 to about 10 mls. In a preferred embodiment, the unit dose applicator is provided as a dispenser having unit dose quantities as discussed above.

A particularly preferred dispenser is the MicroDose™ dispenser commercially available from Unicep Packaging, Inc., Sandpoint, Ind. Alternatively, the dispenser may be an ampule designed to mate with a plunger of a syringe to facilitate controlled delivery of the composition, such as described in U.S. Pat. No. 6,328,715.

As an alternative to the unit dose applicator preferentially utilized as described above, a bottle designed so as to dispense only a certain, measured dose may be used. Alternatively, the composition may be provided in a conventional bottle with instructions to measure a dose, with or without a dedicated appliance for so doing (e.g. cup, syringe). Alternative delivery vessels that do not deliver premeasured quantities of liquid lack the advantages of convenience and higher probability of administration of the correct amount of the composition, but may be more economical than delivery of the composition using a unit dose system.

In one embodiment of the present invention, the composition is preferably administered as a plurality of sublingual applications in order to maximize effective uptake of the active ingredient by the patient, for example, for those whose arthritis symptoms may require more composition than can be conveniently administered in one application for its effective or entire relief. Alternatively, a plurality of unit doses can be administered over a period of days or weeks to patients in need thereof, in order to provide prolonged, relatively low-dose administration of feverfew extract and ginger extract to achieve the desired effective.

When the composition to be administered is a liquid or gel composition, the first unit dose is preferably applied and held in place under the tongue for a predetermined time, preferably about 30 seconds, or more preferably about 60 seconds or more, after which the composition is swallowed. Most preferably, the composition is circulated or “swished” around the mouth by the patient as the tablet disintegrates prior to swallowing. Surprisingly, this apparently minor addition to the procedure noticeably increases the effect of the composition in the treatment. A second unit dose of the composition is then preferably applied and held under the tongue for a predetermined time, preferably about 30 seconds, or more preferably about 60 seconds or more, after which the second composition also is swallowed. Again, preferably the composition is circulated or “swished” around the mouth by the patient prior to swallowing.

The invention will further be described by reference to the following non-limiting representative of some of the examples.

### EXAMPLES

### Example 1

A sub-lingual tablet as generally described above was prepared in 500 mg tablet form, comprising 1.25 mg feverfew extract standardized to 1% parthenolide concentration and 6.25 mg ginger extract standardized to 4% gingerol concentration. The other ingredients in the formulation are common tablet ingredients. The other ingredients in the formulation are Crospovidone, Mannitol, Magnesium Stearate, Silicon Dioxide, Sorbitol, and Natural Flavors.

Samples of these sublingual tablets were given to four individuals for trial evaluation. No adverse events were reported. All test subject reports indicate relief from the symptoms of arthritis or joint pain. In some cases individuals had previously used numerous over-the-counter and prescription medications. Test subjects compared the inventive feverfew/ginger sub-lingual tablets to their previous experience with other medications. Every report found a positive result from the inventive product when compared to the medications that these individuals had used previously.

More specifically:

Subject #1 reported relief from arthritis pain and the discontinuance of an Over-the-counter (OTC) aspirin product with no return of generalized arthritis pain.

Subject #2 reported relief from pain in the neck due to compressed vertebrae after several days of taking the product. This subject had previously endured nearly constant neck pain for several years.

Subject #3 reported that the product “appears to be working.”

Subject #4 reported relief from chronic hip pain, which had persisted for several years. This subject reported relief within three days of taking the feverfew/ginger sublingual tablets.

All patents, patent documents, and publications cited herein are incorporated by reference as if individually incorporated. Unless otherwise indicated, all parts and percentages are by weight. The foregoing detailed description has been given for clarity of understanding only. It will be appreciated that numerous modifications and variations of the invention are possible in light of the above teachings, and therefore the invention may be practiced otherwise than as particularly described.

What is claimed:

1. A method of treating arthritis comprising sublingually administering an effective amount of a composition comprising feverfew extract and ginger extract to a subject in need of such treatment.

2. The method of claim 1, wherein the composition is provided in the form of an orally disintegrating tablet.

3. The method of claim 2, wherein the tablet disintegrates in water in less than about 200 seconds.

4. The method of claim 2, wherein the tablet disintegrates in water in about 10 to about 120 seconds.

5. The method of claim 1, wherein the composition comprises feverfew extract and ginger extract in amount equivalent to 0.5% to 20.0% ginger and 0.010% to 1.0% feverfew on a w/w basis.

6. The method of claim 1, wherein the composition is provided in unit dose form, and comprises from about 0.1 mg to about 5 mg of feverfew extract and from about 1 mg to about 20 mg of ginger extract per unit dose.

7. The method of claim 1, wherein the composition is provided in unit dose form, and comprises from about 0.1 mg to about 3 mg of feverfew extract and from about 3 mg to about 10 mg of ginger extract per unit dose.

8. The method of claim 6, wherein the composition is administered in an administration regimen of from about 1 to about five unit doses in a twenty-four hour period.

9. The method of claim 1, wherein the feverfew extract used for formulating the composition has a standardized parthenolide concentration at initial manufacture of not less than about 0.5%.

10. The method of claim 1, wherein the feverfew extract used for formulating the composition has a standardized parthenolide concentration at initial manufacture of from about 0.8% to about 2.0%.

11. The method of claim 1, wherein the feverfew extract used for formulating the composition has a standardized parthenolide concentration at initial manufacture of about 1%.

12. The method of claim 1, wherein the ginger extract used for formulating the composition has a standardized gingerol concentration at initial manufacture of not less than about 1%,

13. The method of claim 1, wherein the ginger extract used for formulating the composition has a standardized gingerol concentration at initial manufacture of from about 2 to about 20%.

14. The method of claim 1, wherein the ginger extract used for formulating the composition has a standardized gingerol concentration at initial manufacture of from about 2% to about 6%.

15. The method of claim 1, wherein the ginger extract used for formulating the composition has a standardized gingerol concentration at initial manufacture of about 4%.

16. The method of claim 1, wherein the composition contains substantially no active ingredients other than those that are extractable from herbal sources.

17. The method of claim 1, wherein the composition contains substantially no active ingredients other than those that are extractable from feverfew and ginger sources.

18. The method of claim 1, wherein the composition is a liquid/gel composition having a viscosity that is from about 100 cP to about 50,000 cP.

19. The method of claim 1, wherein the composition is a liquid/gel composition having a viscosity that is from about 500 cP to about 5000 cP.

20. The method of claim 1, wherein the composition is held sublingually for at least about 30 seconds prior to swallowing.
